(19) United States
(12) Patent Application Publication
(10) Pub. No.: US 2016/0338874 A1
(43) Pub. Date: Nov. 24, 2016

(54) SYSTEM AND METHOD FOR ASSESSING TISSUE AFTER HYPOTHERMIA

(71) Applicant: Zoll Circulation, Inc., San Jose, CA (US)

(72) Inventors: Uday lllindala, San Jose, CA (US); Richard A. Helkowski, Redwood City, CA (US)

(21) Appl. No.: 14/716,677

(22) Filed: May 19, 2015

(51) Int. Cl. A61F 7/12; A61B 6/00; A61B 5/00; A61B 5/055; A61B 6/03

(52) U.S. Cl. CPC A61F 7/12; A61B 5/055; A61B 6/032; A61B 5/0044; A61B 6/503; A61F 2007/126

(57) ABSTRACT

The amount of granulated tissue, e.g., heart tissue, in a patient who has received therapeutic hypothermia following, e.g., cardiac arrest or myocardial infarction is ascertained using, e.g., magnetic resonance imaging, and based on the amount of such granulated heart tissue, subsequent treatment of the patient is implemented.

### TECHNICAL FIELD

The present application relates generally to systems and methods for assessing tissue, e.g., myocardial tissue, after induced therapeutic hypothermia in, e.g., cardiac arrest (CA) and acute myocardial infarction (AMI) patients.

### BACKGROUND

Patient temperature control systems have been introduced to prevent fever in patients in the neuro ICU due to suffering from sub-arachnoid hemorrhage or other neurologic malady such as stroke. Also, such systems have been used to induce mild or moderate hypothermia to improve the outcomes of patients suffering from such maladies as stroke, cardiac arrest, myocardial infarction, traumatic brain injury, and high intracranial pressure. Examples of intravascular heat exchange catheters are disclosed in U.S. Pat. Nos. 7,914,564, 6,416,533, 6,409,747, 6,405,080, 6,393,320, 6,368,304, 6,338,727, 6,299,599, 6,290,717, 6,287,326, 6,165,207, 6,149,670, 6,146,411,6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, 8,888,729, and USPPs 2013/0178923, 2013/0079855, 2013/0079856, 2014/0094880, 2014/0094882, 2014/0094883, all of which are incorporated herein by reference.

External patient temperature control systems may be used. Such systems are disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference.

In evaluating the efficacy of hypothermia to treat AMI and CA patients using any of the above systems, the outcomes of treated patients may be monitored. For example, because of its widely-noted efficacy, therapeutic hypothermia is now recommended to be considered for CA patients by the American Heart Association, and is recommended standard of care for CA patients according to the International Liaison Committee on Resuscitation (ILCOR).

### SUMMARY

It is believed that therapeutic hypothermia saves tissue, e.g., myocardium (heart tissue), in stricken patients, in particular in AMI and CA patients (post-resuscitation). In understanding how much myocardium is saved, biomarkers such as, e.g., Mason's Tri-Chrome stain, H&E stain, etc. may be used as part of the autopsy of test animals to identify completely spared tissue or, equivalently, to identify necrotic tissue. Similarly, magnetic resonance imaging (MRI)/spectroscopy or positron emission tomography-computed tomography (PET-CT) or equivalent may be used in live patients for the same purposes. Post-hypothermic treatment of human patients can be planned based on such assessments.

As understood herein, therapeutic hypothermia, e.g., in CA and/or AMI patients, may result not only in completely spared heart tissue, but also in granulated tissue, which may be considered to be new formative and/or connective tissue e.g., with small blood vessels. Such granulated tissue, while not in the same condition as completely spared tissue, can help in cardiac recovery and healing. As also understood herein, hypothermic and/or post-hypothermic treatment of human patients may be planned based on an understanding of the amount of granulated tissue in a patient.

Immunohistochemistry biomarkers may be used to determine the efficacy of hypothermia treatment of various tissues in the body, such as cardiac tissue. Biomarkers or markers may be used to identify a specific tissue cell structure which is an indicator of whether or not that cardiac tissue is likely to survive. For example, biomarkers can be used to detect if macrophages are present in the tissue and/or if angiogenesis is taking place in the tissue, which in turn allows for an assessment of the degree of cardiac healing taking place in that tissue, and whether or not hypothermia treatment is effective.

Optionally, the cardiac tissue may be targeted with a drug carried to the tissue site by a particular biomarker or other carrier. Optionally, additional therapeutic hypothermia may be planned based on the amount of observed granulated tissue, including length and temperature of the planned therapeutic hypothermia.

Additionally, enhanced tissue healing post-hypothermia may be provided using a coating (with biomarkers) for timed release in a delivery catheter, or localized release in the target tissue may be provided.

A diagnostic, kit and/or method of providing targeted therapy in conjunction with hypothermia treatment may be provided.

Accordingly, in one aspect a method includes obtaining at least one scan of tissue of a patient on whom therapeutic hypothermia has been implemented. The method also includes ascertaining an amount of granulated tissue in the scanned tissue based on the scan, and implementing treatment of the patient based on the amount of granulated tissue.

In examples, the method may include using magnetic resonance imaging (MRI) to obtain the scan. In other examples, the method may include using computed tomography (CT) to obtain the scan.

In non-limiting embodiments, the method may include ascertaining an amount of granulated tissue in heart tissue based on the scan using a computer implementing image recognition on the scan.

If desired, the method may include determining that the amount of granulated tissue in heart tissue indicates implementing a healing treatment of the patient. The method may include determining that the amount of granulated tissue in the heart tissue indicates implementing a preservation treatment of the patient.

Non-limiting implementations of the method may include implementing the healing treatment including administering to the patient a myocardium healing drug. The healing treatment can include one or more of: administering to the patient cell type-specific drug therapy targeted to granulated tissue in the myocardium, inducing additional therapeutic hypothermia in the patient, and determining a target temperature and/or duration of the additional therapeutic hypothermia based at least in part on the amount of granulated tissue in the heart tissue.

In example embodiments, the method may include, prior to obtaining the scan of heart tissue of the patient on whom therapeutic hypothermia has been implemented, introducing at least one biomarker, e.g., a biomarker or magnetic resonance imaging (MRI) biomarker, into the myocardium of the patient. The biomarker may include one or more of the following: lectin, to bind to new cells; vimentin, to bind to fibroblasts; and CD107A, to bind to macrophages.

If desired, the biomarker can be tagged with at least one metal detectable by an MRI apparatus. The metal can include, e.g., gadolinium and/or manganese. The metal may be bound to at least one contrast agent, and/or the biomarker can be bound to a therapeutic drug. Or, the biomarker may be tagged with a fluorescent tag or dye for imaging.

In some implementations, the method includes indirectly ascertaining an amount of granulated tissue in the heart tissue based on the scan at least in part by ascertaining an amount of imaged necrotic tissue and/or live tissue and inferring an amount of granulated tissue based on the amount of imaged necrotic tissue and/or live tissue.

In other implementations, the method can include directly ascertaining an amount of granulated tissue in the heart tissue based on the scan at least in part by ascertaining an amount of imaged granulated tissue in the at least one scan. The amount of granulated tissue may be ascertained at least in part by ascertaining, e.g., an amount of connective tissue in the scan, and/or by ascertaining an amount of fibroblasts in the scan, and/or by ascertaining an amount of macrophages in the scan.

In example implementations, the method may include determining whether the amount of granulated tissue in the heart tissue satisfies a threshold amount, and based on a determination that the amount satisfies the threshold, indicating a healing treatment. In contrast, based on a determination that the amount does not satisfy the threshold, the method may indicate a preservation treatment different from the healing treatment.

In some embodiments the method can include determining whether a ratio of granulated tissue G to live tissue L satisfies a threshold, and based on a determination that the ratio satisfies the threshold, indicating a healing treatment. In contrast, based on a determination that the ratio does not satisfy the threshold, the method may indicate a preservation treatment different from the healing treatment.

Yet again, the method may include determining whether a ratio of the amount of granulated tissue G to necrotic tissue N satisfies a threshold, and based on a determination that the ratio satisfies the threshold, indicating a healing treatment. On the other hand, based on a determination that the ratio does not satisfy the threshold, the method may indicate a preservation treatment different from the healing treatment.

The threshold may vary according to how much live tissue L and/or necrotic tissue N is present in the heart tissue.

In another aspect, at least one computer memory that is not a transitory signal includes instructions executable by at least one processor for receiving at least one image of a patient's heart, identifying granulated tissue in the image, and determining whether the granulated tissue satisfies a threshold. The instructions are further executable for, responsive to a determination that the granulated tissue satisfies the threshold, outputting an indication that healing treatment is indicated. The instructions are executable for, responsive to a determination that the granulated tissue does not satisfy the threshold, outputting an indication that preservation treatment is indicated.

In another aspect, a catheter that includes at least one distal segment advanceable into a vasculature of a patient. The distal segment includes at least one heat exchange element through which working fluid can flow in a closed circuit to exchange heat with blood flowing past the heat exchange element when the distal segment is advanced into the vasculature to induce therapeutic hypothermia in the patient. At least one chamber is in the distal segment and is configured to convey at least one imaging biomarker into the vasculature of the patient.

The details of the various embodiments described herein, both as to their structure and operation, can best be understood in reference to the accompanying drawings, in which like reference numerals refer to like parts, and in which:

### BRIEF DESCRIPTION OF THE DRAWINGS

FIG. 1 is a schematic view of a non-limiting system in accordance with one embodiment;

FIG. 2 is a series of photographs of heart tissue sections of test subject(s) illustrating, by means of stain testing, spared endocardium, necrotic tissue, and granulated tissue at therapeutic hypothermia temperatures of 32° C. and 35° C. and for comparison at normothermia (38° C.);

FIG. 3 is a schematic diagram showing overall procedure according to present principles;

FIG. 4 is a flow chart of an example process according to present principles;

FIGS. 5 and 6 are diagrams of the distal segments of example intravascular heat exchange catheters that can be used to deliver therapeutic drugs and/or biomarkers to the heart during therapeutic hypothermia;

FIG. 7 is a block diagram of an example computer that may be employed in one or more steps of the logic of FIG. 4:

FIG. 8 is an example output, in this case on a computer display, showing the results of analysis and indicating treatment based thereon;

FIG. 9 is a series of photographs showing the decreasing amount of at-risk myocardium as the target temperature decreases;

FIG. 10 is a series of photographs illustrating spared endocardium (referred to as “live” tissue below), necrotic tissue, and granulation tissue (referred to as “granulated tissue” below), showing the visual differences in appearance between these three general types of tissue;

FIG. 11 includes graphs of infarct size relative to AAR and LV, respectively versus target hypothermic temperature reached, illustrating that with achieving lower target temperature, infarct size decreases;

FIG. 12 includes graphs of infarct size relative to LV and cardiac output versus target hypothermic temperature reached, illustrating that with achieving lower target temperature, infarct size decreases and cardiac output increases;

FIG. 13 includes graphs of infarct size as a percentage of AAR and granulation as a percentage of AAR versus target hypothermic temperature reached, illustrating that with achieving lower target temperature, infarct size decreases and the amount of granulated tissue increases; and

FIG. 14 is a schematic diagram of a kit according to present principles

### DETAILED DESCRIPTION

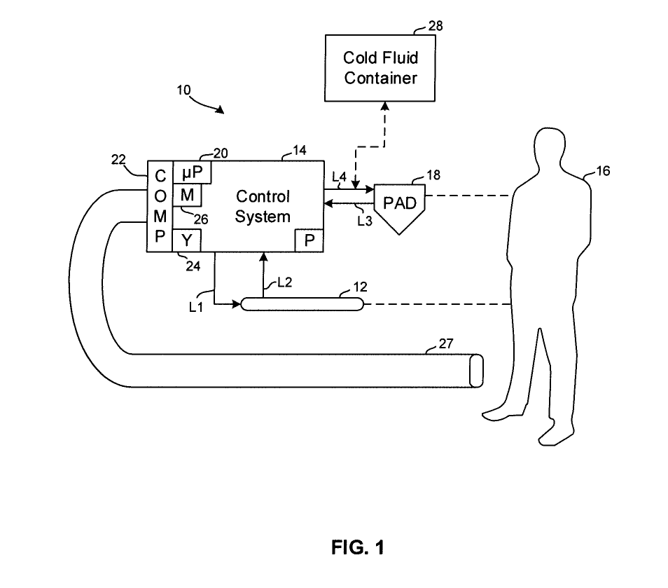

Referring initially to FIG. 1, in accordance with present principles, a system 10 may include an intravascular heat exchange catheter 12 controlled by a control system 14 to control patient temperature, e.g., to prevent the patient 16 from becoming febrile or to induce therapeutic hypothermia in the patient 16. In the catheter, working fluid or a coolant such as but not limited to saline circulates (typically under the influence of a pump “P” in the control system) in a closed loop from the control system 14, through a fluid supply line L1, through the catheter 12, and back to the system 14 through a fluid return line L2, such that no working fluid or coolant enters the body. While certain preferred catheters are disclosed herein, it is to be understood that other catheters can be used in accordance with present principles, including, without limitation, any of the catheters disclosed above or in the following U.S. patents, all incorporated herein by reference: U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, 8,888,729, 5,486,208, 5,837,003, 6,110,168, 6,149,673, 6,149,676, 6,231,594, 6,264,679, 6,306,161,6,235,048, 6,238,428, 6,245,095, 6,251,129, 6,409,747, 6,368,304, 6,338,727, 6,299,599, 6,287,326, 6,126,684, 7,211,106 and USPPs 2013/0178923, 2013/0079855, 2013/0079856, 2014/0094880, 2014/0094882, 2014/0094883, all of which are incorporated herein by reference. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by the control system 14 or other control system to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia. The pad 18 may receive working fluid from the system 14 through a fluid supply line L3, and return working fluid to the system 14 through a fluid return line L4. The pump “P” may be a peristaltic pump which may engage any one of the lines L1-L4, which are typically plastic or other material IV lines, to urge working fluid through the lines through peristalsis.

The control system 14 may include one or more microprocessors 20 receiving target and patient temperatures as input and controlling, among other things, the pump “P” and a refrigerant compressor 22 and/or a bypass valve 24 that can be opened to permit refrigerant to bypass a condenser. The processor 20 may access instructions on one or more computer memories 26 such as but not limited to solid state and disk-based memory to execute temperature management protocols based on the target temperature (typically input by an operator into the system 14) and feedback from a patient temperature sensor (on the catheter, or on an esophageal probe, rectal probe, tympanic measurement device, etc.)

Other external/internal modes for inducing therapeutic hypothermia in patients may be used. In some embodiments, a patient suffering from CA and/or AMI may be cooled as rapidly as possible or at some other optimized rate to 32° C.

While various embodiments described herein refer to methods and systems for assessing, analyzing and/or ascertaining the granulated tissue present in cardiac or heart tissue and implementing treatment of the patient based on the detected granulated tissue, it is contemplated that such methods and systems may be utilized to assess, analyze or ascertain granulated tissue in various tissue types, e.g., organ tissue, vascularized organ tissue, brain tissue, and other tissues in the body. Also, in certain embodiments, the amount, volume, density or other characteristic or parameter of the granulated tissue may be assessed, analyzed or ascertained.

Also, in various embodiments described herein, biomarker may refer to any molecule used to determine the general state (as in threshold) or a measurable indicator of a state (prevalence) based on the expression of a defined molecule. Biomarkers may include, e.g., antibodies, proteins, stains, molecules, substances, markers or any biological or chemical compound. Biomarkers may bind to or otherwise couple to various tissues, cells and/or cell structures or may be taken up by a cell.

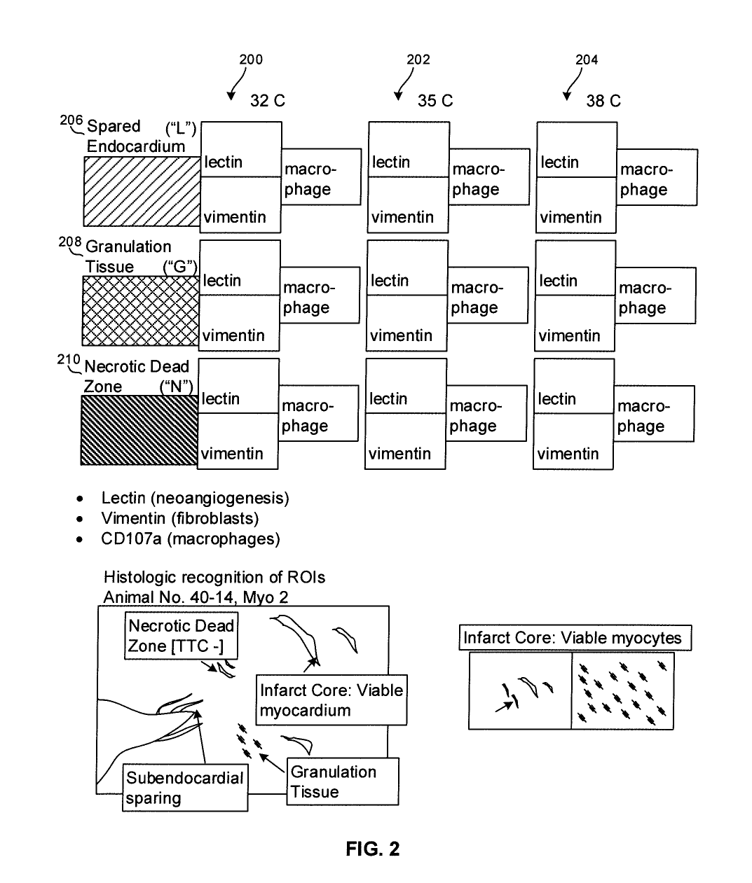

FIG. 2 illustrates photographs of heart tissue after therapeutic hypothermia has been induced to cool the tissue to 32° C. (at 200), 35° C. (202), and normothermia 204. Spared endocardium 206 (“L”) is shown in the top row of photographs, granulated tissue 208 (“G”) is shown in the middle row of myocardium photographs, and necrotic (dead) tissue 210 (“N”) is shown in the bottom row of myocardium. Granulated tissue G can include, e.g., connective tissue, macrophages, and fibroblasts. For each tissue type at each temperature, photographs are provided showing when two stains, in this case, lectin and vimentin (to identify connective tissue, new or reforming tissue, angiogenesis, and/or fibroblasts), are used to reveal the tissue structure, as well as macrophage tissue stained in this case using a CD107a marker. The photographs show that more endocardium is spared at the lowest temperature than at the intermediate temperature, which in turn shows more spared tissue than normothernmia, with the same being true of granulated tissue 208. Necrotic tissue is correspondingly reduced.

The above three biomarkers, were chosen to identify and quantify granulated tissue. Lectin stain identifies new expression in cells and hence angiogenesis. Vimentin identifies fibroblasts as they are the first stage of connective tissue and cardiac tissue repair. Macrophages are the clean up after apoptosis and are quantifiable in healing tissue with no signal present in normal or unharmed myocardium, endocardium or other cardiac tissue.

Accordingly, it will be appreciated that in FIG. 2, immunohistochemical staining using lectin (for angiogenesis), vimentin (for fibroblasts), and CD107a (for macrophages) are used to discriminate granulated tissue from “dead” necrotic myocytes. Primarily, the granulation zone (present in all three groups) is larger in the 32° C. group than the other two groups. There is spared myocardium in all three groups and is clearly dose (temperature) dependent. The spared endocardium in the 32° C. group has vessel walls still intact and no signal from any of the biomarker stains. The spared myocardium in the 38° C. group has some macrophage activity as the spared parts are quite close to the damaged myocardium as well (204). The 32° C. group (200) has the highest stain signal from all three stains in the granulated zone and the necrotic zone. The signal strength may be high due to the large section that is healing (granulated) and a necrotic zone is much smaller with the granulated tissue surrounding the region (hence a strong signal). There progressively is a larger region in the “healing” and “clean up” stage in the 32° C. group than the 35° C. or 38° C. groups. The biomarkers may be tagged with a dye (or fluorescence) and when the biomarker (e.g., lectin or vimentin) is taken up in the cell or attached to it, it will show a signal. The signal may be counted/scanned or measured for florescence.

Present principles understand that heretofore, the presence of granulated tissue amongst the potentially damaged myocardium tissue in CA or AMI patients who have undergone therapeutic hypothermia has not been recognized as pointing to a better survival of the tissue after repair. Instead, prior to present principles, treatment has been to monitor patient recovery by broad signals such as cardiac ejection fraction. Using present principles to detect the prevalence of granulated tissue post-therapeutic hypothermia, an option is provided to directly treat the granulated tissue to promote healing thereof, as the channels for nutrients delivery to damaged areas are still viable. This can potentially give the physician an immediate pharmacological therapy option targeting the healing pathway (using, e.g., available pharmacokinetic treatments like cortisone) as opposed to simply limiting risk and damage (e.g., through antiplatelet therapy, blood thinners, etc.) Such healing treatment can provide a better outcome for patients that present significant granulation.

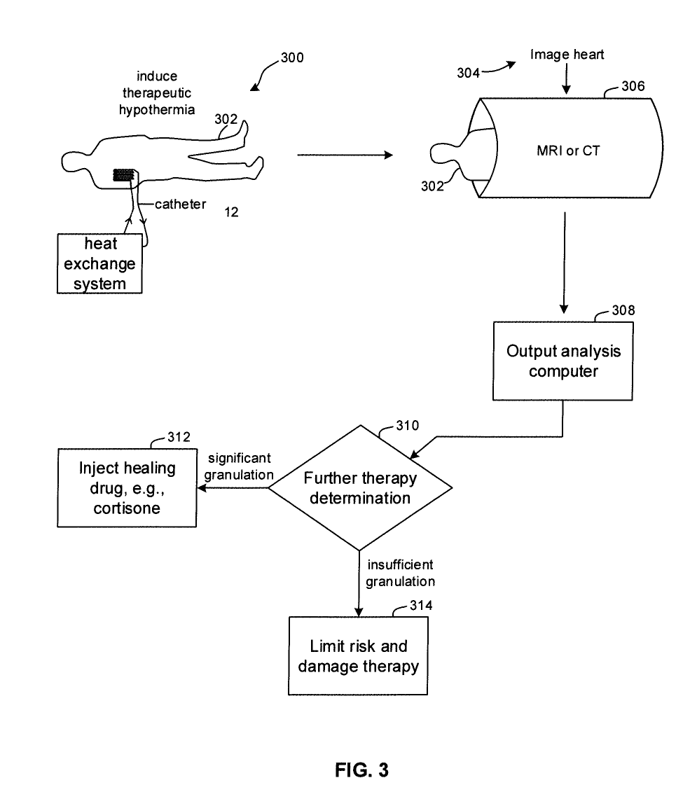

Now referring to FIG. 3, at 300 therapeutic hypothermia is induced into a patient 302 suffering from, e.g., AMI or CA (post-resuscitation), using the intravascular heat exchange catheter 12 connected to the heat exchange control system 14. Alternatively or in addition, the external pad 18 may be used to induce hypothermia. Therapeutic hypothermia preferably is induced as soon as possible post-resuscitation and as quickly as possible to target temperature.

Hypothermia is induced to lower the patient's temperature to below 38° C., more preferably to below 35° C., and more preferably still to 32° C. to maximize the amount of heart tissue that is preserved from necrosis. Among the above-incorporated patent documents, details of various protocols for inducing hypothermia in a patient are discussed.

Post-hypothermia, at 304 the amount and type of tissue in the heart of the patient 12 is assessed using imaging afforded by an MRI or CT apparatus 306 in which all of most of the body of the patient 302 is disposed, after having introduced or during introduction of appropriate scanning apparatus biomarkers (e.g., lectin, vimentin or CD107a with an MRI detectable metal coupled thereto), or another suitable biomarker into the patient. The heart images are output to an analysis computer 308 and analyzed at 310, programmatically if desired according to algorithms examples of which are disclosed below, to make further therapy determinations. For example, if the heart images indicate that a clinically significant amount of granulated tissue exists, healing therapy may be executed at 312, such as injecting into the patient a healing drug such as cortisone. Cell type-specific drug therapy can be targeted to the tissue of interest. On the other hand, if a clinically insufficient amount of granulation is determined to exist from the images of the heart, then preservation therapy may be executed at 314 to limit risk and further damage to the heart (e.g., using antiplatelet therapy, blood thinners, etc.).

In addition, the healing therapy may include inducing additional therapeutic hypothermia in the patient after analyzing for granulated tissue on the ground that hypothermia therapy is working and should be continued. Or, the healing therapy may include not inducing additional hypothermia on the ground that hypothermia has worked and need not be continued. Healing therapy may also include, in the event that additional therapeutic hypothermia is indicated, a new target temperature. For example, in the presence of a relatively large amount of granulated tissue, a relatively higher (but still hypothermic) target temperature may be indicated for follow-on therapeutic hypothermia, whereas in the presence of a relatively small amount of granulated tissue, a relatively lower target temperature may be indicated for follow-on therapeutic hypothermia.

Similarly, preservation therapy may include inducing additional therapeutic hypothermia in the patient after analyzing for granulated tissue on the ground that hypothermia therapy has not yet worked as well as was intended. Or, the preservation therapy may include not inducing additional hypothermia on the ground that hypothermia has not worked at all in the present patient and need not be continued.

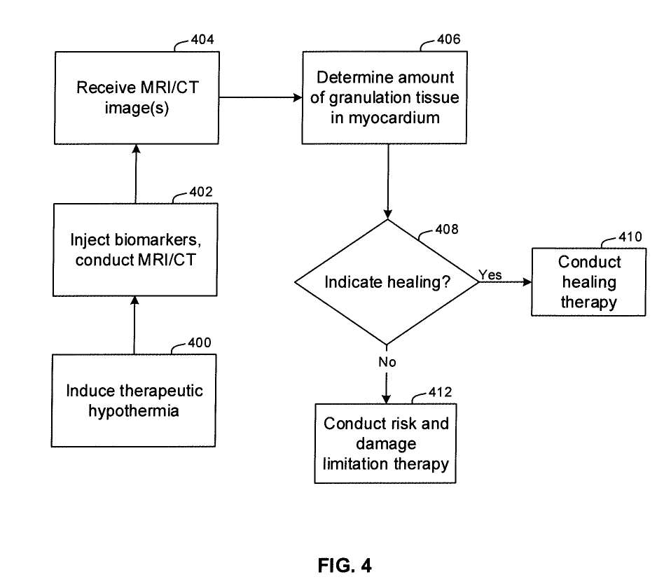

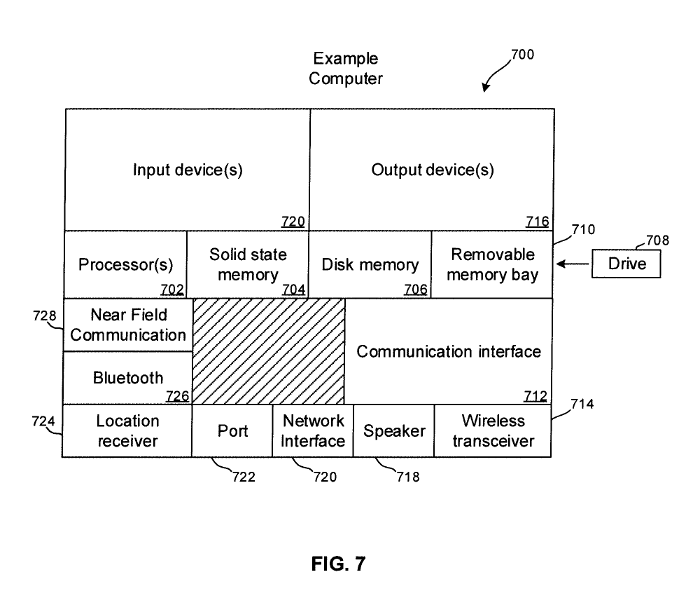

FIG. 4 illustrates the above principles further. Note that one or more of the steps in FIG. 4 may be executed by a computer or combination of computers, each of which may include the appropriate components shown in FIG. 7 and described further below. One or more steps may require human intervention, e.g., to commence the introduction of a therapeutic hypothermia regime.

Commencing at block 400, therapeutic hypothermia is induced in a patient such as a CA or AMI patient for a suitable period, e.g., for 2-6 hours or other appropriate period, preferably as soon as possible post-resuscitation. At block 402, MRI biomarkers may be introduced and MRI imaging or CT imaging or other appropriate myocardium imaging conducted. Imaging typically is conducted at the conclusion of hypothermia treatment but may be conducted while the patient remains hypothermic or undergoes hyperthermia treatment.

In an example, the following biomarkers may be used: lectin (to bind to new cells, also referred to herein as “new or reforming tissue”, which cells indicate angiogenesis), and/or vimentin (to bind to fibroblasts common to connective tissue), and/or CD107A (to bind to macrophages). These biomarkers bind to the tissue of interest, namely, granulated or spared granulated myocardium, cardiac tissue or other tissue that has the potential to heal. The biomarkers may be tagged with metals that are connected directly or indirectly to the biomarkers. The metals are detectable in the MRI apparatus shown in FIG. 3. The metals may include, e.g., gadolinium and manganese. These metals may typically be furthermore bound to a contrast agent because alone they can be toxic. Manganese is a preferred metal because of its relatively smaller size and cell uptake compared to gadolinium.

The images as augmented by the biomarkers are received at block 404 and then at block 406 the amount of granulated tissue in the heart is determined. In the images, white areas produced as a result of reflection off of the metal that is bound to the biomarkers are visible as indicators of the amounts of the respective granulated tissue types for which respective biomarkers have been introduced into the patient. That is, the metal is bound to the biomarker, which in turn binds to or is taken up by the tissue of interest, thereby allowing identification of the tissue of interest in the images and, through image analysis, detection of how much of the tissue of interest exists. To state again, the granulated tissue of interest that can be identified in this way exists in this particular state as a result of therapeutic hypothermia.

The determination at block 406 may be executed by, e.g., a computer within the MRI apparatus 306, or by, e.g., the analysis computer 308 receiving, over a wired or wireless communication path, myocardium images from the MRI apparatus 306. Other modes of making the determination at block 406 may be employed.

To make the determination at block 406, the amount of granulated tissue in the image(s) from the MRI (or CT) apparatus 306 is determined directly or indirectly. While only a single image may be used and, hence, the “amount” of granulated tissue refers to an area in a single cross-section, typically, the amount of granulated tissue may be determined by adding together the areas of granulated tissue depicted in multiple images to effectively determine a volume of granulated tissue in the heart. The amount of all three of the above-described species of granulated tissue (connective tissue, macrophages, and fibroblasts) may be used, or the amount of only one of the above-described species of granulated tissue may be used, or the amount of only two of the above-described species of granulated tissue may be used. When multiple species of granulated tissue are used, the area encompassed by each species in each image is added to the areas encompassed by each species in the remaining images.

Image recognition may be used to identify granulated regions for the above purposes. In one example, electronically stored templates are provided for each of the above-described species of granulated tissue, and the processor of the analyzing computer matches elements of the images to the templates to determine the amount of granulated tissue. Image recognition encompasses signal recognition such that signals from respective biomarkers or markers provided for each tissue type (live tissue L, necrotic tissue N, and granulated tissue G) may be compared to expected signals for that tissue type to determine the amount of the respective tissue in the heart. However, biomarkers may be provided only for granulated tissue G if desired.

To directly measure granulated tissue, an appropriate biomarker or biomarkers may be injected into the patient to provide a signal representative of granulated tissue G, and the signal or image then analyzed to determine, from the biomarker signal, how much granulated tissue is in the heart. Appropriate biomarkers for live tissue L and necrotic tissue N, such as, respectively, biomarkers bound to Manganese and Gadolinium, may also be injected into the patient to provide signals in each MRI or CT image representative of the amounts of their respective tissues, although this is not required. The biomarker(s) for granulated tissue G may be a combination of visible biomarkers and MRI-sensitive markers.

To measure the amount of granulated tissue indirectly, the above-described biomarkers for live and necrotic tissue L, N may be injected into the patient and the respective signals measured, and then subtracted from the total amount of tissue in the image of the heart to yield a remainder which may be inferred to be the amount of granulated tissue.

Regardless of how the amount of granulated tissue is determined, the process moves to decision diamond 408 to determine whether healing therapy is indicated for implementation at block 410 or whether risk/damage mitigation therapy is indicated for implementation at block 412.

The determination at decision diamond 408 may be made using one or more algorithms. For example, if the amount of granulated tissue satisfies (as by meeting or exceeding) a (typically non-zero) threshold amount, healing may be indicated. Otherwise, risk/damage mitigation therapy is indicated.

Or, the ratio of the amount of granulated tissue G to live tissue L may be used, and if the ratio satisfies (as by meeting or exceeding) a (typically non-zero) threshold ratio, healing may be indicated. Otherwise, risk/damage mitigation therapy is indicated. In other embodiments, the opposite algorithm may be used: if the ratio satisfies (as by being less than) a threshold ratio, healing may be indicated.

Yet again, the ratio of the amount of granulated tissue G to necrotic tissue N may be used, and if the ratio satisfies (as by meeting or exceeding) a (typically non-zero) threshold ratio, healing may be indicated. Otherwise, risk/damage mitigation therapy is indicated. In other embodiments, the opposite algorithm may be used: if the ratio satisfies (as by being less than) a threshold ratio, healing may be indicated.

The threshold amount or ratio of granulated tissue above which healing therapy is indicated may vary according to how much live tissue L and/or necrotic tissue N is present. Thus, for example, with relatively little live tissue L, an amount of granulated tissue G may have to satisfy a relatively low (typically non-zero) threshold amount to result in an indication of healing, whereas for a relatively large amount of live tissue L, an amount of granulated tissue G may have to satisfy a relatively high (typically non-zero) threshold amount to result in an indication of healing. The above variances may be reversed.

Similarly, with relatively little live tissue L, a ratio of granulated tissue G to live tissue L may have to satisfy a relatively high (typically non-zero) threshold ratio to result in an indication of healing, whereas for a relatively large amount of live tissue L, a ratio of granulated tissue G to live tissue L may have to satisfy a relatively low (typically non-zero) threshold ratio to result in an indication of healing. The above variances may be reversed.

Yet again, with relatively little necrotic tissue N, an amount of granulated tissue G may have to satisfy a relatively high threshold amount to result in an indication of healing, whereas for a relatively large amount of necrotic tissue N, an amount of granulated tissue G may have to satisfy a relatively low (typically non-zero) threshold amount to result in an indication of healing. The above variances may be reversed.

Similarly, with relatively little necrotic tissue N, a ratio of granulated tissue G to necrotic tissue N may have to satisfy a relatively high threshold ratio to result in an indication of healing, whereas for a relatively large amount of necrotic tissue N, a ratio of granulated tissue G to necrotic tissue N may have to satisfy a relatively low (typically non-zero) threshold ratio to result in an indication of healing. The above variances may be reversed.

Still further, with relatively little necrotic tissue N, a ratio of granulated tissue G to live tissue L may have to satisfy a relatively high threshold ratio to result in an indication of healing, whereas for a relatively large amount of necrotic tissue N, a ratio of granulated tissue G to live tissue L may have to satisfy a relatively low (typically non-zero) threshold ratio to result in an indication of healing. The above variances may be reversed.

The quantification of the live or granulated tissue cells may be expressed as percent of the left ventricle (LV) volume and that is not at the cell level but at the region level and the prognostic value may be evaluated (based on previous MRI studies) quite precisely.

In an example, the area of necrotic tissue caused by CA or AMI may be about 30% of the left ventricle (LV) with an infarct of about 50%, meaning that the dead area would be about 15% of LV. With hypothermia most of the dead tissue may be salvaged into granulated tissue, leaving about 1% of the LV necrotic. 1% may be hard to detect clearly. But live tissue L and/or granulated tissue G would be about 14% of LV, so depending on the degree of granulation the therapy may be to give active healing dosage as opposed to the limiting risk option.

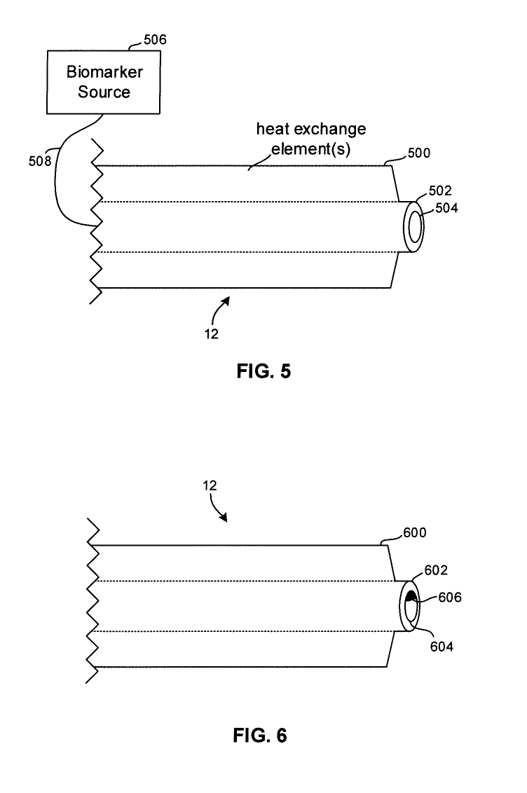

FIG. 5 shows the distal portion of a heat exchange catheter 12 embodied as any one of the catheters disclosed in the relevant incorporated patent documents as modified herein in which one or more heat exchange elements 500 are provided to exchange heat with blood flowing past the heat exchange element 500, through which element working fluid such as saline flows in a closed loop such that heat exchange is effected through the wall of the heat exchange element, and no blood enters the working fluid and no working fluid enters the blood. The catheter 12 has a distal end 502 at which a through-lumen 504 terminates. The through-lumen 504 may be a radially central guide wire lumen.

A source 506 of biomarker agent/healing agent may communicate with the through-lumen 504 via a line such as an IV line 508 to inject a biomarker, and/or healing substance such as cortisone into a patient in whom the catheter 12 is disposed. In an example, a MRI-sensitive biomarker is attached or bonded to a metal as described above and optionally to a healing drug targeted for the patient. In addition, the biomarker may be injected into the patient along with a healing drug. Optionally, the healing drug may be delivered using a separate carrier.

FIG. 6 shows the distal portion of an alternate heat exchange catheter 12 embodied as any one of the catheters disclosed in the relevant incorporated patent documents as modified herein in which one or more heat exchange elements 600 are provided to exchange heat with blood flowing past the heat exchange element 600, through which element working fluid such as saline flows in a closed loop such that heat exchange is effected through the wall of the heat exchange element, and no blood enters the working fluid and no working fluid enters the blood. The catheter 12 has a distal end 602 at which a through-lumen 604 terminates. The through-lumen 604 may be a radially central guide wire lumen. An elution chamber with elution detection substance 606 in the through-lumen 604, for example, may elute a detection substance that adheres to live/granulated tissue only. Optionally, the substance may be bound with a targeted healing drug. The detection substance 606 may be eluted at the end of the cooling process and imaged thereafter, e.g., about four hours later, to obtain a picture or signal of the degree of alive tissue, granulated tissue or healing tissue.

FIG. 7 shows an example computer 700 components of which may be part of the MRI or CT apparatus 306 and/or analysis computer 308.

In general, the computer 700 may operate with a variety of operating environments. For example, operating systems from Microsoft, or a Unix operating system, or operating systems produced by Apple Computer or Google may be used.

The computer 700 may include one or more processors 702 executing instructions that configure the device to receive and transmit data over a network such as a wireless network. The computer 700 may be instantiated by a personal computer, a server, a laptop computer, a personal digital computer, a wireless telephone, etc.

Information may be exchanged over a network between network devices. To this end and for security, devices can include firewalls, load balancers, temporary storages, and proxies, and other network infrastructure for reliability and security. One or more devices may form an apparatus that implement methods of providing a secure community such as an online social website to network members.

As used herein, instructions executable by the processor 702 such as the instructions described in reference to FIG. 4 above refer to computer-implemented steps for processing information in the system. Instructions can be implemented in software, firmware or hardware and include any type of programmed step undertaken by components of the system.

A processor may be any conventional general purpose single- or multi-chip processor that can execute logic by means of various lines such as address lines, data lines, and control lines and registers and shift registers.

Software modules described by way of the flow charts and user interfaces herein can include various sub-routines, procedures, etc. Without limiting the disclosure, logic stated to be executed by a particular module can be redistributed to other software modules and % or combined together in a single module and/or made available in a shareable library.

Present principles described herein can be implemented as hardware, software, firmware, or combinations thereof; hence, illustrative components, blocks, modules, circuits, and steps are set forth in terms of their functionality.

Further to what has been alluded to above, logical blocks, modules, and circuits described below can be implemented or performed with a general purpose processor, a digital signal processor (DSP), a field programmable gate array (FPGA) or other programmable logic device such as an application specific integrated circuit (ASIC), discrete gate or transistor logic, discrete hardware components, or any combination thereof designed to perform the functions described herein. A processor can be implemented by a controller or state machine or a combination of computing devices.

The functions and methods described below, when implemented in software, can be written in an appropriate language such as but not limited to C# or C++, and can be stored on or transmitted through a solid state memory 704 such as a random access memory (RAM), read-only memory (ROM), electrically erasable programmable read-only memory (EEPROM), and/or through a disk memory 706 such as a compact disk read-only memory (CD-ROM) or other optical disk storage such as digital versatile disc (DVD), magnetic disk storage or other magnetic storage devices including removable thumb drives 708 engageable with removable memory bays 710, etc. A connection may establish a computer-readable medium. Such connections can include, as examples, hard-wired cables including fiber optics and coaxial wires and digital subscriber line (DSL) and twisted pair wires. Such connections may include wireless communication interfaces 712 including infrared and radio interfaces. Note that a non-transitory computer readable storage medium explicitly includes hardware such as flash memory which may lose data upon loss of power.

The computer 700 may include one or more wireless telephony transceivers 714 that may confirm to standards such as but not limited to Mobitex Radio Network, DataTAC, GSM (Global System for Mobile Communication), GPRS (General Packet Radio System), TDMA (Time Division Multiple Access), CDMA (Code Division Multiple Access), CDPD (Cellular Digital Packet Data), iDEN (integrated Digital Enhanced Network), EvDO (Evolution-Data Optimized) CDMA2000, EDGE (Enhanced Data rates for GSM Evolution), UMTS (Universal Mobile Telecommunication Systems), HSDPA (High-Speed Downlink Packet Access), IEEE 802.16e (also referred to as Worldwide Interoperability for Microwave Access or “WiMAX)” orthogonal frequency division multiplexing (OFDM).

The example computer 700 may (but not must) include one or more output devices 716 such as a printer or video display that may be implemented by a high definition or ultra-high definition “4K” or higher flat screen and that may be touch-enabled for receiving user input signals via touches on the display. The computer 700 may include one or more speakers 718 for outputting audio in accordance with present principles, and at least one input device 720 such as, e.g., a point and click device, key entry device, and an audio receiver/microphone for e.g. entering audible commands to the computer 700 to control the computer 700. The example computer 700 may also include one or more network interfaces 720 for communication over at least one network under control of one or more processors 702. Thus, the interface 720 may be, without limitation, a Wi-Fi transceiver, which is an example of a wireless computer network interface. It is to be understood that the processor 702 controls the computer 700 to undertake present principles, including the other elements of the computer 700 described herein such as e.g. controlling the output device 716 to present images thereon and receiving input therefrom. Furthermore, note the network interface 720 may be, e.g., a wired or wireless modem or router, or other appropriate interface such as, e.g., a wireless telephony transceiver, or Wi-Fi transceiver as mentioned above, etc.

In addition to the foregoing, the computer 700 may also include one or more input ports 722 such as, e.g., a high definition multimedia interface (HDMI) port or a USB port to physically connect (e.g. using a wired connection) to another computing device and/or a headphone port to connect headphones to the computer 700 for presentation of audio from the computer 700 to a user through the headphones.

Also in some embodiments, the computer 700 can include one or more position or location receivers 724 such as but not limited to a cellphone receiver, GPS receiver and/or altimeter that is configured to e.g. receive geographic position information from at least one satellite or cellphone tower and provide the information to the processor 702.

Also included on the computer 700 may be a Bluetooth (including low energy Bluetooth) transceiver 726 and other Near Field Communication (NFC) element 728 for communication with other devices using Bluetooth and/or NFC technology, respectively. An example NFC element can be a radio frequency identification (RFID) element.

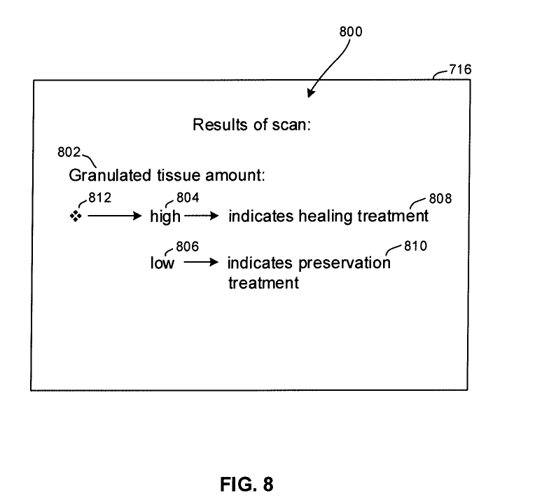

FIG. 8 shows that the output device 716, when implemented as a display, may present a user interface (UI) 800 that includes the result of the MRI or CT scan and presents indicated treatment options based thereon. At 802 the UI indicates that it is presenting an indication of the amount of granulated tissue in the patient. In the non-limiting example shown, at 804 a high amount is indicated. If desired, at 806 the UI 800 may also indicate a low amount. At 808 the UI 800 presents a perceptible message that the healing treatment is indicated for the high amount 804, whereas at 810 the UI 800 presents a perceptible message that the preservation treatment is indicated for the low amount 806. Specific types of healing treatment and preservation treatment described above may be part of the indications 808, 810, respectively.

Should it be determined, e.g., that the amount of granulated tissue determined in block 406 of FIG. 4 satisfies a threshold amount at decision diamond 408, an indication 812 may appear informing that the image reveals that the high amount of granulated tissue is present. It is to be understood that in such a case, the low indicator 806 may be presented for informational purposes or may not appear at all on the UI 800.

Similarly, should it be determined, e.g., that the amount of granulated tissue determined in block 406 of FIG. 4 fails to satisfy the threshold amount at decision diamond 408, the indication 812 may point to the low amount indicator 806. It is to be understood that in such a case, the high indicator 804 may be presented for informational purposes or may not appear at all on the UI 1800. In the example shown, the indicator 812 may include an asterisk and an arrow as shown, pointing to the high indicator 804. Colors may also be used, e.g., the indicator 812 may be a different color, for example red, than the rest of the UI 800, which may be in black and white.

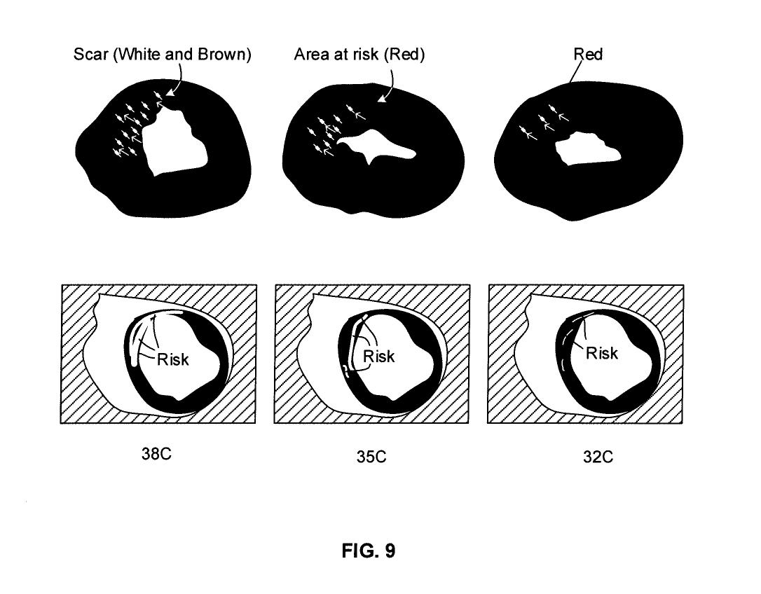

FIGS. 9-13 illustrate in various ways that the lowest feasible hypothermic target temperature that may be induced without risking heart failure, approximately 32° C., provides the greatest patient benefit in terms of reduced infarct size, at-risk tissue, improved post-therapy cardiac output, and improved post-therapy granulated tissue amounts.

As shown in FIG. 9 from left to right, the amount of cardiac tissue that is at-risk after CA or AMI decreases from the amount of at-risk tissue that is present when no hypothermic treatment is used (left-most photographs) to the amount of at-risk tissue that is present after hypothermic treatment at 35° C. (middle photographs), which in turn is greater than the amount of at-risk tissue when a target temperature of 32° C. is reached (right-most photographs).

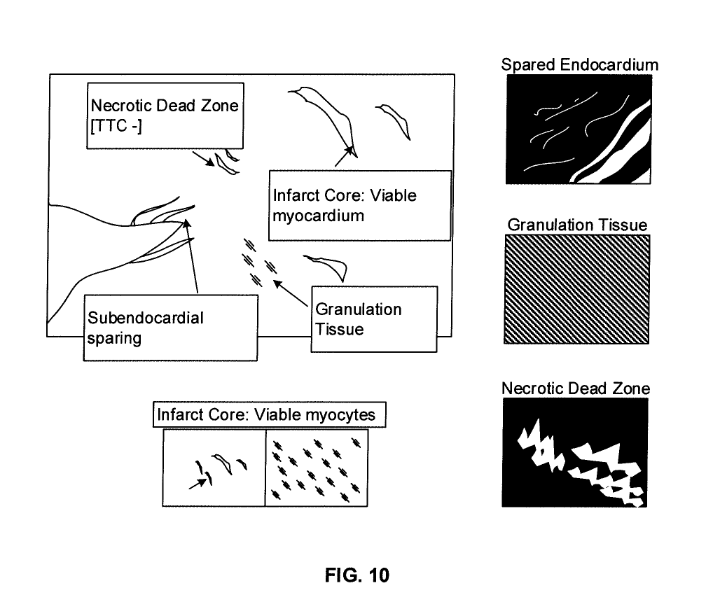

FIG. 10 is a series of photographs illustrating spared endocardium (referred to as “live” tissue above), necrotic tissue, and granulation tissue (referred to as “granulated tissue” above), showing the visual differences in appearance between these three general types of tissue.

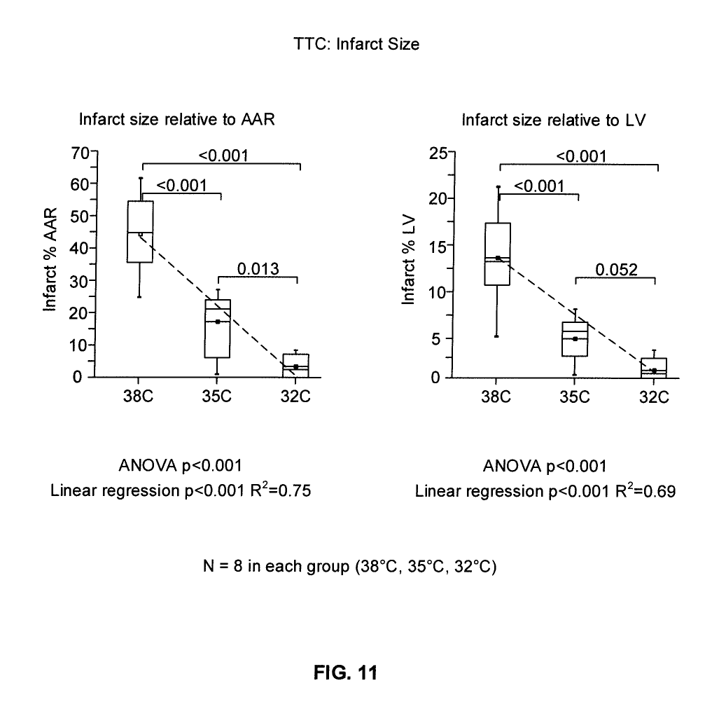

FIG. 11 shows graphs of infarct size relative to AAR (left) and LV (right) versus target hypothermic temperature reached, illustrating that with achieving lower target temperature, infarct size decreases using both measures.

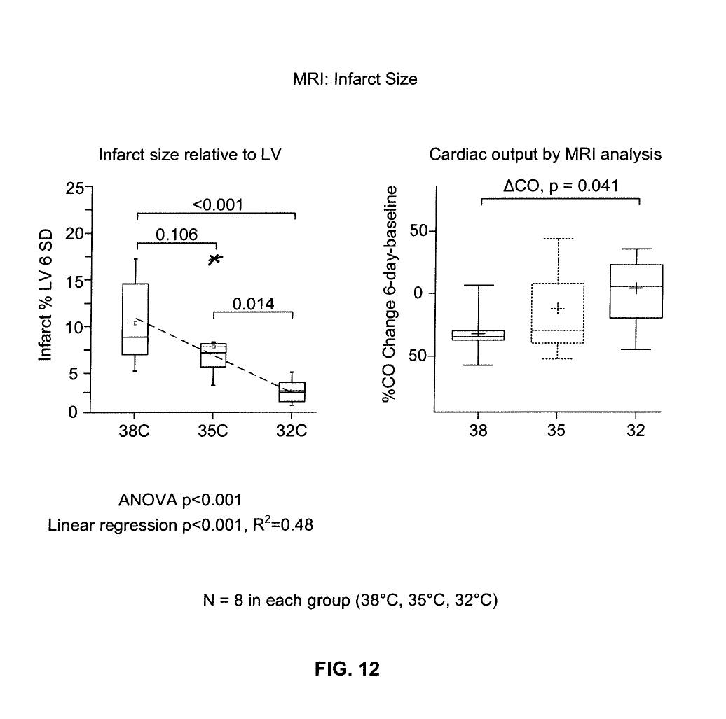

FIG. 12 includes graphs of infarct size relative to LV (left) and cardiac output (right) versus target hypothermic temperature reached, illustrating that with achieving lower target temperature, infarct size decreases and cardiac output increases, in some cases almost linearly between 38° C. and 32° C.

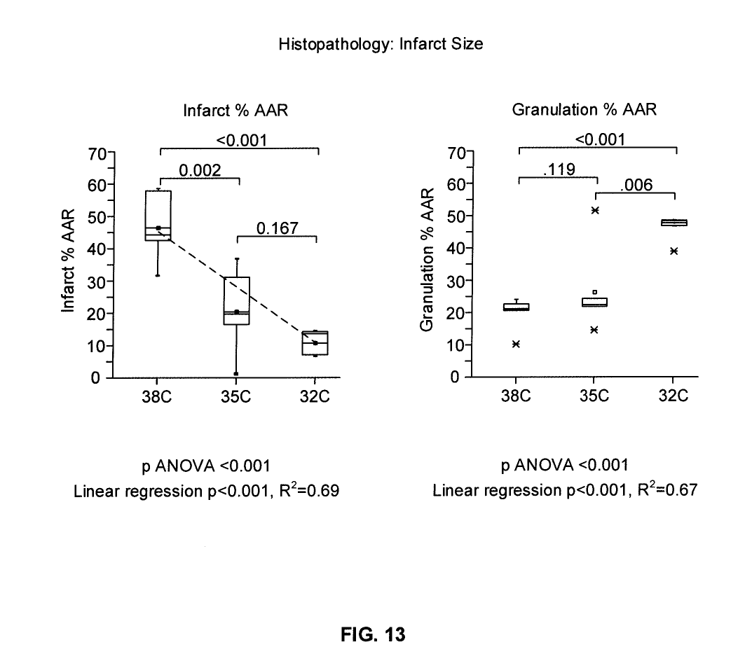

FIG. 13 includes graphs of infarct size as a percentage of AAR (left) and granulation as a percentage of AAR (right) versus target hypothermic temperature reached, illustrating that with achieving lower target temperature, infarct size decreases almost linearly between 38° C. and 32° C. and the amount of granulated tissue increases significantly when comparing the amount of such tissue when target temperature is only 35° C. with the significantly greater amount of such tissue, potentially treatable as described above, when target temperature of 32° C. is reached.

While the above discussion focuses on evaluating granulated tissue in the myocardium to determine treatment, the same principles may be used to evaluate granulated tissue in other vascularized organs including the liver, kidney, and brain.

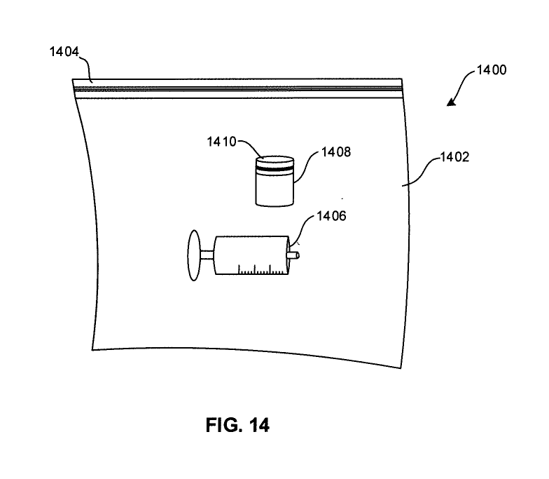

FIG. 14 shows a diagnostic kit 1400 that includes a diagnostic holder 1402 which may be established by, e.g., a plastic bag with a closure 1404 such as a zip-loc closure to provide access to the interior of the holder 1402. Within the holder is at least one syringe 1406 and at least one closable container 1408 containing any of the above-mentioned biomarker substances 1410 for detecting granulated tissue in a patient on whom therapeutic hypothermia has been implemented. The biomarker may be detectable by an imaging apparatus.

Components included in one embodiment can be used in other embodiments in any appropriate combination. For example, any of the various components described herein and/or depicted in the Figures may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

While various embodiments are herein shown and described in detail, the scope of the present invention is to be limited by nothing other than the appended claims.

1. A method comprising:
obtaining at least one scan of tissue of a patient on whom therapeutic hypothermia has been implemented;
ascertaining an amount of granulated tissue in the tissue of the patient based on the scan; and
implementing treatment of the patient based on the amount of granulated tissue.

2. The method of claim 1, comprising using magnetic resonance imaging (MRI) or computed tomography (CT) to obtain the scan.

3. (canceled)

4. The method of claim 1, comprising ascertaining an amount of granulated tissue in the tissue of the patient based on the scan using a computer implementing image recognition on the scan.

5. The method of claim 1, comprising determining that the amount of granulated tissue in the tissue of the patient indicates implementing a healing treatment of the patient.

6. The method of claim 1, comprising determining that the amount of granulated tissue in the tissue of the patient indicates implementing a preservation treatment of the patient.

7. The method of claim 5, comprising implementing the healing treatment including administering to the patient a myocardium healing drug.

8. The method of claim 5, comprising implementing the healing treatment including administering to the patient cell type-specific drug therapy targeted to granulated tissue in the myocardium.

9. The method of claim 5, comprising implementing the healing treatment including inducing additional therapeutic hypothermia in the patient.

10. The method of claim 9, comprising determining a target temperature of the additional therapeutic hypothermia based at least in part on the amount of granulated tissue.

11. The method of claim 1, comprising, prior to obtaining the at least one scan of tissue of the patient on whom therapeutic hypothermia has been implemented, introducing at least one biomarker into the patient, which binds to the granulated tissue.

12. The method of claim 11, wherein the biomarker includes lectin to bind to new cells, vimentin to bind to fibroblasts or CD107A to bind to macrophages.

13-14. (canceled)

15. The method of claim 11, wherein the biomarker is tagged with at least one metal detectable by an MRI apparatus.

16-19. (canceled)

20. The method of claim 1, comprising indirectly ascertaining an amount of granulated tissue in the tissue of the patient based on the scan at least in part by ascertaining an amount of imaged necrotic tissue and/or live tissue and inferring an amount of granulated tissue based on the amount of imaged necrotic tissue and/or live tissue.

21. The method of claim 1, comprising directly ascertaining an amount of granulated tissue based on the scan at least in part by ascertaining an amount of imaged granulated tissue in the at least one scan.

22. (canceled)

23. The method of claim 1, comprising ascertaining the amount of granulated tissue at least in part by ascertaining an amount of connective tissue in the at least one scan.

24. The method of claim 1, comprising ascertaining the amount of granulated tissue at least in part by ascertaining an amount of fibroblasts in the at least one scan.

25. The method of claim 1, comprising ascertaining the amount of granulated tissue at least in part by ascertaining an amount of macrophages in the at least one scan.

26-30. (canceled)

31. The method of claim 1, comprising determining whether a ratio of granulated tissue G to live tissue L satisfies a threshold, and based on a determination that the ratio satisfies the threshold, indicating a healing treatment, and based on a determination that the ratio does not satisfy the threshold, indicating a preservation treatment different from the healing treatment.

32. The method of claim 1, comprising determining whether a ratio of the amount of granulated tissue G to necrotic tissue N satisfies a threshold, and based on a determination that the ratio satisfies the threshold, indicating a healing treatment, and based on a determination that the ratio does not satisfy the threshold, indicating a preservation treatment different from the healing treatment.

33. The method of claim 1, comprising determining whether granulated tissue in the heart tissue satisfies a threshold, the threshold varying according to how much live tissue L and/or necrotic tissue N is present in the heart tissue.

34-62. (canceled)
